(19) United States
(12) Patent Application Publication
(10) Pub. No.: US 2018/0312554 A1
(43) Pub. Date: Nov. 1, 2018

(54) METHOD TO HARVEST INSECTS, EGGS, AND LARVAE

(71) Applicant: LAVACA RIVER RANCH, LTD., Houston, TX (US)

(72) Inventor: Walter G. Mayfield, Houston, TX (US)

(73) Assignee: LAVACA RIVER RANCH, LTD., Houston, TX (US)

(21) Appl. No.: 15/965,876

(22) Filed: Apr. 28, 2018

Related U.S. Application Data
(60) Provisional application No. 62/491,567, filed on Apr. 28, 2017.

(51) Int. Cl. C07K 14/435; A61K 35/64; C07K 1/14; A23J 1/08

(52) U.S. Cl. CPC C07K 14/43563; A23J 1/08; C07K 1/145; A61K 35/64

(57) ABSTRACT

This process or components of this process may be used as a method of harvesting and processing for food consumption for many types of insects in various stages of development. Also the process may be used as a dual purpose for both harvesting protein and restoration of land for such invasive insects like red fire ants and their associated fire ant mounds.

### BACKGROUND OF THE DISCLOSURE

### 1. Field of Disclosure

The present disclosure relates to a method for harvesting insects, insect eggs, insect pupae, and larvae. The present disclosure particularly relates to such a method wherein the insects being harvested are ants and the subject ants routinely move their eggs and larvae above ground such as fire ants.

### 2. The Related Art

Going back to the time of the Aztecs, ant larvae has been harvested for food consumption. To-day in Mexico, the ant larvae and pupae are better known as escamoles. Escamoles are considered a Mexican delicacy like caviar and are seasonal with harvesting being generally in April and May from the species Liometopum apiculatum and L. occidentale var. luctuosu. They are harvested from the roots of the Agave tequilana (tequila) or Agave americana (maguey-mezcal) plants in Mexico but can be also found under cacti in Southern U.S.

They taste buttery and nutty, with a texture akin to that of cottage cheese, are very nutritious and high in protein. Re-ported prices can range between $30 to $100 per kilo due to the limited number of ant colonies for the above described species, the need to carefully manage the ant colonies for long term production and the labor-intensive hand harvesting methods used by harvesters in Mexico. Other types of ant eggs from a variety of species are eaten around the world and are considered a delicacy.

### SUMMARY OF THE DISCLOSURE

In one aspect, the invention is a method for harvesting insects, eggs, pupae, and larvae comprising collecting above surface insect mounds, and separating the insects, eggs, pupae, and larvae from the mound materials and other materials co-collected.

In another aspect, the invention is a composition having antibiotic, antifungal, and other desirable properties prepared by the process of collecting fire ant eggs, pupae, and larvae from fire ant mounds, and using an aqueous solution producing from the fire ant eggs, pupae and larvae an organic composition having antibiotic, antifungal and other desirable properties.

In still another aspect, the invention is a food source comprising ants and/or grasshoppers.

### DETAILED DESCRIPTION OF THE DISCLOSURE

Just a few restaurants in the U.S. serve escamole due to its scarcity and because much of the U.S. culture is unaccustomed to eating insects. A solution to the scarcity problem of ant eggs as food is a method to harvest ant larvae on a large scale using both labor and mechanical means.

Not limiting this process to a particular type of ant larvae or insect, in the U.S. there is an abundance of an invasive species of red ants native to South America known as red imported fire ants or Solenopsis invicta which are medium-sized red and black colored ants that build mounds of soft soil. Mounds are rarely larger than 18″ in diameter and mound numbers can range between 200 to 800 per acre. The ants were imported on ships from most likely from Brazil and have no natural predators in the US.

In their native home Brazil and Latin America, the phorid fly belonging to the family Phoridae prays on the red fire ant limiting its population. The phorid fly did not arrive with the red fire ant to the U.S., although attempts are being made to artificially introduce it.

In the U.S., the invasive fire ants and their ant mounds wreak havoc on ranchers, farmers, and city dwellers with billions of dollars spent on chemical killing agents. The ants kill quail, deer, lizards, songbirds, horn toads, and most any small living creature along with the insects that provide food for native species. The invasive fire ant has now spread to most of the southern United States and Mexico and there is concern with them becoming further adaptive to colder northern climates.

Fire ant queens can live for up to 7 years and lay over 1000 eggs a day. The eggs grow into larvae which are fed, then grow into pupae, then develop into mature red ants. The eggs, larvae, and pupae generally are off colored white.

With the world population growing and in need of additional protein sources, a method has been needed to harvest insects along with their larvae, pupae, and eggs which is less labor intensive, more mechanical, and less expensive than current methods used in Mexico and especially from a source such as the invasive red ant that is substantially more abundant. Additionally, with a more economic method to harvest invasive red fire ants along with their protein rich eggs, lar-vae, and pupae, more can be done to control their populations in an economic fashion that wreaks havoc on the environment in the US, Mexico, China and other countries where red fire ants have spread and where few natural predators exist.

In Mexico the escamole harvesters will typically hand dig ants and their eggs underneath various plants leaving a portion of the ant colony still intact for future harvest. The harvesters will typically hand pick the individual escamole pupae, larvae, and eggs out of the dirt and ants. They will wash the escamole and sell as a delicacy to local Mexican restaurants.

In the Southern US, the invasive red imported fire ant of the genus Solenopsis invicta builds ant mounds out of dirt creating numerous tunnels that extend far underground. The eggs, pupae, and larvae (hereinafter referred to as eggs) will be placed in various locations around the ant mounds most likely due to temperature. In the morning or evenings, the eggs may be found within the side of the mound facing the sun to capture additional temperature for optimal growth environment.

The ants have many different types of workers including those who tend for the eggs. The ants are extremely protective of their eggs as can be seen when an ant mound is disturbed, they will take their eggs to lower levels as a priority to protect the eggs from the sunlight and predators. When rain causes a flood which overwhelms the ant mound, the ants will float together along with their eggs of which examples can be seen on YouTube or other films and photos. Once the ants floating on water reach the shore, they will carry their eggs along with the queens and will propagate its new ant mounds.

Ant mounds may have many queens, and mounds are additionally propagated by a queen leaving to a new location at which she will dig a tunnel in the ground and start laying eggs. Ants love to start new mounds in areas of disturbed soils such as southern U.S. cattle ranches where the cows disturb the soil, eat down the grass, and spread manure all of which creates a more favorable environment for the invasive red fire ant. A thousand acre cattle pasture may have as many as 800,000 ant mounds.

As help towards a solution to control the invasive red fire, to increase the supply of edible protein and restoration of ranch land, the following is a method to harvest insects and eggs including those from the invasive red fire ant.

A laborer with shovel or a mechanical piece of equipment such as a tractor with front end loader, scoop, back hoe, back hoe with thumb escalator, back how with front hoe combination, vacuum, mower with leaf and debris collecting system, device similar to snow auger with blower to holding container, clamshell grabs, finger grabs, loader with tooth bar, grass fork bucket, bucket with teeth, tipping buckets, grapple buckets, mulcher with bucket, grapple with bucket, plow, booster bucket, tree spade, other types of spades, a multi sided bucket that simultaneously pinches more than one side of the ant mound as a scoop, a bucket that can slice under the mound with any type of back stop to assist the mound to enter the bucket, or other similar de-vices or combinations of such can be used to assist in the harvesting of the ant mounds productive with eggs and ants. They can also be used and equipment may be modified to harvest just a particular part of the ant mound to maximize the productivity of harvesting the most eggs with least amount of ant mound and other debris.

Typically, ant mounds will have grass with roots that make a simple bucket harvest difficult due to the grass roots causing the ant mound to flop over as the bucket blade slices below the ant mound which can scatter the mound along with the valuable ant eggs and ants in front of and outside the bucket. Therefore, additional methods may be used to capture the mound and eggs to further load into a transport vehicle for further processing. Additionally, a conveyor belt can be added to a transport device such as a tractor, wagon, dump bed, trailer, or other transport device that can assist in moving the ant mound from the ground to a holding bed or tank for processing or transport.

Additionally, while the eggs and ants are harvested, the tractor can dual task by using the front end loader or a back blade, disk, plow or other accessories to knock down the ant mounds to smooth out the field as an additional benefit to the rancher, farmer, or land owner.

Additionally, when the Mound Material is placed in a container filled with liquid such as water, a milky whitish looking liquid will rise to the surface. This milky looking material most likely derives from the ants, eggs, larvae, or pupae and is most likely used for food, antimicrobial, antifungal, or for other beneficial purposes for the ants. The milky looking material may have beneficial uses to mankind including medicinal, antifungal, antimicrobial, topical cream, food, or other beneficial purposes. The method and process used herein may also apply to harvesting of the milky looking substance that floats to the surface.

After the ants and eggs along with unwanted debris such as dirt, grass, sand, nuts, dead insects, and anything else associated with an ant mound hereinafter described as Mound Material are loaded in a transport vehicle, the Mound Material is then transported to a processing and separation facility. Note that the transport device may include processing and separation equipment.

The inventor has found that on one particular ranch in Texas during the month of March with an outside temperature of 78 degrees with many new ant mounds having been recently built, in 10 minutes five cubic feet of the prime part of a productive ant mound with ants and eggs could be harvested into a front end loader with the help of a shovel. After separation, four pounds of ants and eggs were harvested of which about 30 percent by volume were eggs alone. The ant eggs when first born are very small and can be less than ½ millimeter in length. By the time they grow into pupae, they can be around 3 to 5 millimeters in length and 1 to 3 millimeters in width. Although, various types and species of ant eggs, larvae, and pupae may be of different sizes.

The processing and separation facility is used to separate the unwanted debris from the valuable ants and eggs in the Mound Material. It may be further used to separate the ants from the eggs wherein just ants may be sold or just eggs may be sold. Also, the ant mounds may have special properties such as enriched dirt or mulch that may be further sold and utilized for gardens or fertilizer. In addition to the eggs being sold as food, the eggs and the ants may have medicinal qualities or may be used in insect killer baits, pet food, skin creams, dietary supplements, or for any other purposes or purposes yet to be discovered.

When the Mound Material is brought to be processed for separation, several methods may be used. Some of the debris of the Mound Material is of the same size as the ants and eggs and can float in liquid such as water along with the ants and eggs making separation difficult. If needed, a crushing device may be used to crumble the mounds and separate large debris such as unwanted grass.

A front end loader, shaker, shovel, bull dozer or most any device can crush the mounds and crumble the larger clumps if needed. Separation may begin with a water bath or with various mesh sized sieves. Either the bath or sieves can be used first. The bath may include a holding tank, bucket, pond, hole, hole with plastic lining or other type of water holding device for the Mound Material in which water may be added. A stirring method may be used that includes a shovel or any automated mechanical device that acts to stir including a rotating cement truck like tank or a stirring or blender object inside the tank. Also, vibrating devices, compressed air, gravity, centrifugal, forces, or air bubbles may be used both with the sieves and tank to further assist in separation.

The eggs and ants along with wood, grass, nuts and other debris will typically float, but some of the eggs and ants will stick in the bottom mud until stirred. Some of the eggs and ants can be separated from the bottom mud by pouring the bottom mud through sieves of various sizes and pressure washing the mud through the sieve with water. As the eggs and ants and other debris float to the surface of the water tank with Mound Material that has been stirred, a small mesh sized sieve can be used to extract ants and eggs from the water surface or a large sieve or rake may be used at this point to clear away the larger debris such as grass, sticks, and wood from the water surface. A smaller mesh sieve may then be used to separate the eggs and ants from the water. The smaller mesh sieve used to extract the eggs, ants, grass, and other debris from the water should have a sieve mesh size smaller than the circumference of the eggs and ants. The eggs and ants may have a length longer than their width, so the sieve mesh size should be smaller than their width. If the smaller mesh sieve is first used, then much of the grass and larger debris may be further removed from the ants and eggs with a larger mesh sized sieve in which the ants and eggs are washed through the larger sized mesh into a container leaving the larger debris in the mesh.

Once the eggs, ants, and similar sized debris have been separated from the larger debris, additional methods will be needed for further separation. Jetting high pressure water on the sieves may help further separate debris.

Another method to assist to separate ants from eggs involves killing the ants while they are still in the Mound by suffocation or a chemical that will not harm food.

Additionally, one particular preferred method involves:

1) Scooping up ant mounds in a water holding container

2) Adding water to the container

3) Stirring the Mound Material and water to loosen the eggs and ants from the Mound Material so the eggs and ants will float to the surface

4) Using a mesh strainer with a smaller mesh size than the eggs to scoop up the floating ants “A”, eggs “E” , and debris “D” or “ AED” collectively

5) Placing AED material which may have large debris included in a large container to air dry the AED. AED may be placed on absorbent material like a towel.

6) Air dried AED may be shaken with a shaker or rolled in a barrel such as a rolling cement bar-rel to further unstick the debris. Towels or absorbent material may be added to barrel for added drying.

7) Ants may then be killed in a food grade manner by suffocation, or a food grade chemical. One method to kill ants is to place AED into ziplock bag. Add food gas without oxygen such as CO2 to the ziplock. Dry ice or container with compressed CO2 may be used.

8) AED may then be run through a conventional optical, color and or shape, integrity sorter to fully separate eggs from ants a debris or eggs and ants from debris or eggs from ants from debris.

9) Eggs and larvae may be refrigerated and transported to be sold as fresh or may be frozen conventionally, or by an number of super cooled methods such as using liquid nitrogen or CO2.

Additionally, another particular preferred method involves:

1) Scooping up ant mounds in a front end loader

2) Adding water to the front end loader

3) Stirring the mud and water to loosen the eggs and ants from the Mound Material.

4) Using a mesh strainer with a smaller mesh size than the eggs to scoop up the floating ants “A”, eggs “E” , and debris “D” or “ AED” collectively

5) Placing AED material which may have large debris included in a large mesh container such as a mesh barrel that rolls and/or shakes so the smaller AED falls through the mesh leaving unwanted debris.

6) Placing resulting AED in a mesh barrel with smaller mesh size than the eggs wherein the mesh barrel rolls and/or shakes to dry the eggs and unstick from ants and debris

7) Using a dehydrator, dehumidifier, heater, air blower or a combination thereof to assist drying while mesh barrel is rotating and or shaking to dry the eggs

8) Drying may continue until outside of eggs and ants are relatively dry, but eggs are still hydrated and fresh in appearance or at some point of partial or total dehydration

9) Ants may then be killed in a food grade manner by suffocation, or a food grade chemical

10) Refuting AED may then be run through a conventional optical, color and or shape, integrity sorter to fully separate eggs from ants a debris or eggs and ants from debris or eggs from ants from debris.

Also, to help to unstick the eggs from the ants and other Mound Material, common food grade anti caking agents may be added such as but not limited to food grade diatomaceous earth, sodium aluminosilicate, calcium carbonate, or magnesium carbonate, or a host of other readily found common food additives to help prevent caking.

Further separation of ants, eggs, and smaller debris may be achieved by additional methods. The inventor has found that a bright light either by sun or artificial on the live ants will cause the live ants to move the eggs to a dark spot such as under a sheet of paper which helps to separate the ants and eggs from dead and other organic debris. Clean dirt, sand, or other easily separated material may be added back in which the live ants can transport the eggs away from the unwanted debris into more easily separated material. Alternatively, the eggs, ants, and smaller debris may be added to a common type ant farm hobby type home with tubes and various living areas or a homemade box configuration or etc in which the live ants may move the eggs to a move favorable location for easier harvesting of eggs from unwanted debris.

In another method of separating the ants and eggs from other debris, the ants either in the Mound Material or later in the process when the eggs and ants are separated from the Mound Material, are killed so not to cause further problems by using one or more of several different means. However it is important to use methods to maintain a food grade product. Methods to kill ants may include drowning with liquid like water, creating an oxygen free environment, or use of some type of chemical or gas that can be later evacuated that does not cause problems with food. Some types of food grade chemicals such as orange extract, lemon extract, citrus extract, vinegar, or other food ingredients may act as deterrents to drive live ants away from the eggs. Additionally, ants may be killed by boiling water, freezing, shaking, tumbling, or high heat. Refrigeration or freezing should be used when needed for preservation of the eggs. When wet, a problem exists with the ants, eggs, and other small debris sticking together.

Once the ants have been killed or live ones driven away, as a start to the separation of the eggs from the dead ants, the moisture should be reduced to help prevent sticking of the eggs and ants by air drying, the sun, compressed air, cooking, an oven, drying by heat, hot air, fan, blow dryer, dehydrator, refrigerator with dehydrator, a chemical addition, or by other means. Various types of clean dirt, sand, powders, or other drying agents may be added to help dry and unstick the dead ants and debris from the sticky eggs. Additionally, a centrifugal force device similar to that in a cloths washing machine or dryer may be used to spin the ant, egg, and debris material for further drying to help unstick. To further help unstick the ants from the eggs which tend to stick together in clumps or ball, a shaking or other process may be used to separate the eggs free of the ants including shaking on a surface including a screen surface, rolling in a tumbler, blasting with compressed gas such as air, or other methods. Using a vertical or horizontal separator with shaking, air flow, gravity dropping, gravity dropping through matrix, using the air frictional drag and weight properties of ants with their legs verse the rounder less drag properties of the eggs to separate via gravity or air flow, using the stickiness factor of the eggs verse ants to adhere to a like type compound that attracts the egg over the ant or vice versa, using centrifugal force, or the better rolling quality of eggs vs ants wherein the eggs will roll away from the ants via a force like gravity or compressed air may be used to help separate the ants from the eggs. Also, eggs can bounce better than ants, so a shaking method can bounce eggs higher than ants and away from ants.

Additionally, to help sort the eggs and or the ants and other debris, an optical sorter including a color optical sorter like the Satake PIKASEN Alpha (FMS-2000) (trademarked) or any variety of the common conventional optical sorters may be used to separate the eggs from other debris or the ants from other debris using color, shape, size, integrity, or a host of other sorting properties that may be visually detected by a machine. The optical sorter may be used at any time along the process from separation of Mound Material without the use of water or it can be used for separation at a later processed stage of the eggs from ants and perhaps some other unwanted associated debris. Typically, the optical sorters will electronically photograph every piece moving at high speeds through the machine. An object such as unwanted debris that does not fit the criteria may be removed during the process by mechanical means including a small precise blast of air. Optical sorters may accurately separate food grade material at many thousands of pounds per hour and are capable of processing large sized food products such as potatoes to the smallest food sized products such as sesame seeds. They are also used to separate different colors and shapes of rice which are not too different from the size and color of ant eggs.

Once desired separation of the ants and eggs is complete, further washing and other good manufacturing principles may be necessary to comply with local rules and regulation for food grade compliance. Additionally, refrigeration and/or freezing and the addition of food grade preservatives and packaging that enhances long range food preservation such as freeze drying or state of the art food packaging may be used. The eggs and ants may be cooked, freeze dried, dehydrated, canned with water or oil based liquid, and herbs, spices, and other ingredients may be added. Popular escamole dishes include plain boiled or pan fried escamole, garlic and chopped onion addition, soups, tacos, chalupa, egg rolls, sprinkled on top of salads or most any types of food groups.

We claim:

1. A method for harvesting insect protein comprising collecting surface ant mounds during a period when ant eggs, ant larvae, and ant pupae are present in the surface mounds.

2. The method of claim 1 further comprising crushing the ant mounds to produce ant mound material comprising ant eggs, ant larvae, ant pupae, adult ants, unwanted debris, and ant mound soil.

3. The method of claim 1 further comprising employing water to concentrate the insect protein by washing the ant mound material with water thereby dissolving water soluble materials; and collecting the ant eggs, ant larvae, ant pupae, adult ants, and unwanted debris floating on the surface of water.

4. The method of claim 3 further comprising stirring the ant mound material prior to collecting the ant eggs, ant larvae, ant pupae, adult ants, and unwanted debris floating on the surface of water.

5. The method of claim 3 further comprising passing the ant eggs, ant larvae, ant pupae, adult ants, and unwanted debris collected from the surface of the water through sieves to reduce the amount of unwanted debris and adult ants present to produce an insect protein concentrate.

6. The method of claim 5 further comprising employing a food safe method to kill or immobilize adult ants present in the insect protein concentrate.

7. The method of claim 5 further comprising passing the insect protein concentrate through an optical color sorter to selectively remove any material that is not ant eggs, ant larvae, and ant pupae.

8. The method of claim 1 further comprising passing the ant mound material through sieves to reduce the amount of unwanted debris, ant mound soil, and adult ants present in the ant mound material.

9. The method of claim 8 further comprising employing water to concentrate insect protein present in the mound material which passed through the sieves by washing the sieved ant mound material with water thereby dissolving water soluble materials; and collecting the ant eggs, ant larvae, ant pupae, adult ants, and unwanted debris floating on the surface of water.

10. The method of claim 9 further comprising stirring the ant mound material prior to collecting ant eggs, ant larvae, ant pupae, adult ants, and unwanted debris floating on the surface of water.

11. The method of claim 9 further comprising employing a food safe method to kill adult ants present in the insect protein.

12. The method of claim 9 further comprising passing the ant eggs, ant larvae, ant pupae, adult ants, and unwanted debris through an optical color sorter to selectively remove any material that is not ant eggs, ant larvae, and ant pupae.

13. The method of claim 1 wherein the surface ant mounds are collected using a mechanism selected from the group consisting of a mechanized bucket with a scoop, vacuum, snow blower, clamshell type bucket, and combinations thereof

14. A method for producing an antibiotic and/or antifungal composition comprising washing fire ant eggs, pupae and larvae with water to produce an emulsion, breaking the emulsion, and collecting the non-continuous phase.

15. A method for harvesting insect protein comprising collecting ants and/or grasshoppers and immobilizing them and then employing an optical color sorter to remove anything not an ant and/or grass hopper from the collected ants and/or grasshoppers.
